SEQUENCE LISTING (10 sequences)

SEQ ID NO: 1 - DNA, 55 bp, artificial
  Forward DNA MYCN 3UTR integration primer
gccaagaagggcggcaagatcgccgtgtaaacgcttctcaaaactggacagtcac

SEQ ID NO: 2 - DNA, 49 bp, artificial
  Reverse DNA MYCN 3UTR integration primer
cttaatgcgccgctacagggcgcgtggccccccaaccaggattgtacag

SEQ ID NO: 3 - DNA, 19 bp, artificial
  Forward primer for MYCN 3UTR in E.coli
cgcaagatccgcgagattc

SEQ ID NO: 4 - DNA, 18 bp, artificial
  Reverse primer for MYCN 3UTR in E.coli
gcaagtgtagcggtcacg

SEQ ID NO: 5 - DNA, 21 bp, artificial
  First oligonucleotide for obtaining pGL4.26-MYCN plasmid
ctagaaagtataatcgataag

SEQ ID NO: 6 - DNA, 21 bp, artificial
  Second oligonucleotide for obtaining pGL4.26-MYCN plasmid
gatccttatcgattatacttt

SEQ ID NO: 7 - DNA, 25 bp, artificial
  First forward primer for nested PCR for amplification of CMV promoter
ctagcaaaataggctgtccccagtg

SEQ ID NO: 8 - DNA, 22 bp, artificial
  First reverse primer for nested PCR for amplification of CMV promoter
cacaccacgatccgatggtttg

SEQ ID NO: 9 - DNA, 24 bp, artificial
  Second forward primer for nested PCR for amplification of CMV promoter
cgttacataacttacggtaaatgg

SEQ ID NO: 10 - DNA, 22 bp, artificial
  Second reverse primer for nested PCR for amplification of CMV promoter
gaagtactcggcgtaggtaatg

(19) United States
(12) Patent Application Publication
(10) Pub. No.: US 2013/0203822 A1
(43) Pub. Date: Aug. 8, 2013

(54) COMPOUNDS FOR TREATMENT OF TUMORS BEARING DEREGULATED MYC ONCOPROTEINS

(71) Applicants: Alessandro Quattrone, Trento (IT); Viktoryia Sidarovich, Trento (IT); Valentina Adami, Trento (IT)

(72) Inventors: Alessandro Quattrone, Trento (IT); Viktoryia Sidarovich, Trento (IT); Valentina Adami, Trento (IT)

(21) Appl. No.: 13/806,880

(22) PCT Filed: Jun. 22, 2011
(86) PCT No.: PCT/IB2011/052735

§ 371 Date: Feb. 19, 2013

(30) Foreign Application Priority Data
Jun. 23, 2010 (IT) TO2010A000539

(51) Int. Cl. C07D 213/89

(52) U.S. Cl. CPC C07D 213/89
USPC 514/348; 514/345

(57) ABSTRACT

Compound of Formula (I): wherein R¹, R² and R³, which are identical or different, are hydrogen atom or C₁₋₄ alkyl, and R⁴ is a saturated C₆₋₉ linear, branched or cyclic hydrocarbon radical or a radical of Formula (II) wherein X is S or O, Y is a hydrogen atom or up to 2 halogen atoms, Z is a single bond or a divalent radical being O, S, —CR₂—, in which R is hydrogen or C₁₋₄ alkyl, or other divalent radical with 2-10 carbon atoms and, optionally, O and/or S atoms linked in the form of a chain, wherein—if the radicals contain 2 or more O and/or S atoms—the latter are separated from one another by at least 2 carbon atoms, and it also being possible for 2 adjacent carbon atoms to be linked together by a double bond, and the free valencies of the carbon atoms being saturated by a hydrogen atom and/or C₁₋₄ alkyl groups, Ar is an aromatic ring system which has up to two rings and which may be substituted by up to three radicals from the group of fluorine, chlorine, bromine, methoxy, C₁₋₄ alkyl, trifluoromethyl and trifluoromethoxy, salts and/or solvates thereof, for use in the treatment of a tumor bearing deregulated MYC oncoproteins, wherein said compound is capable of increasing UTR-dependent expression of at least one MYC gene.

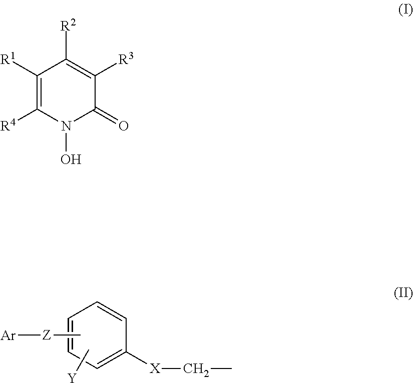

### FIELD OF THE INVENTION

This disclosure concerns compounds for treatment of tumors bearing deregulated MYC oncoproteins.

### BACKGROUND OF THE INVENTION

Oncogene activation is a frequent molecular event in both solid tumors and leukemias and lymphomas. It can be produced by different molecular lesions, the most common being gene dosage increase or amplification, chromosomal translocation, point mutation, promoter or enhancer sequence epigenetic alterations, 5′ untranslated regions (5′UTRs) and 3′ untranslated regions (3′UTRs) alterations.

MYC proteins (MYC, MYON, and MYCL) are basic helix-loop-helix transcription factors involved in the regulation of processes controlling many if not all aspects of cell fate. It is therefore not surprising that these genes are also powerful oncogenes, and represent key lesion points in human cancer, being deregulated by virtually all the above mentioned mechanisms of alterations.

MYC genes are controlled at the transcriptional and posttranscriptional levels in their expression, being the latter essentially the levels of mRNA stability in the cytoplasm and mRNA availability to translation. These two controls are specifically exerted through a number of cis-acting signals residing mainly in the 5′UTR and 3′UTR of the three genes. In several cases the alterations reported in MYC genes in cancer affect these gene regions, further demonstrating the crucial role of post-transcriptional controls of MYC members in tissue homeostasis.

Among the MYC family, MYCN was initially identified as a gene tandemly amplified in 20% of the cases of neuroblastoma, the most frequent paediatric extra-cranial solid tumor. About 35-40% of the patients bearing this alteration have, despite intensive multimodal therapy, a bad prognosis: MYCN amplification and consequent overexpression (not MYCN overexpression without amplification, see PMID: 16510605) is a strong independent predictors of advanced tumour stage, tumor progression and poor outcome, irrespective of concomitant genomic lesions.

MYCN is also found to be over-expressed in cases of other cancers of neural origin, including glioblastoma, medulloblastoma, retinoblastoma, small cell lung carcinoma, primitive neural ectodermal tumors, as well as in some other embryonal tumors.

As it also happens for patients bearing tumors with deregulated activity of the other members of the MYC family, patients with MYCN alterations display therefore remarkably aggressive tumors, which are largely refractory to treatment.

Several attempts have been done in the past to address MYC proteins as key cancer targets, with the proposal of compounds suppressing their activity. But being the category to which these proteins belong, transcription factors, basically undruggable, new ways of dealing pharmacologically with the deregulated expression of MYC family genes are highly expected.

All the preexisting attempts at targeting MYC genes expression rather than MYC proteins activities have been directed to MYC mRNAs, with the aim of suppressing their production or of favoring their degradation.

### SUMMARY OF THE INVENTION

Taking into account these premises, the need is therefore felt for improved solutions enabling the treatment of tumors bearing deregulated, preferably upregulated, MYC oncoproteins.

The object of this disclosure is providing such improved solutions.

According to the invention, the above object is achieved thanks to the subject matter recalled specifically in the ensuing claims, which are understood as forming an integral part of this disclosure.

An embodiment of the present disclosure provides compounds of formula (I):

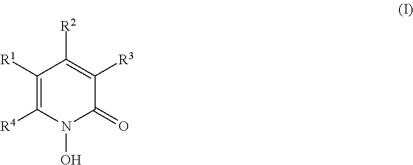

wherein
R¹, R² and R³, which are identical or different, are hydrogen atom or C₁₋₄ alkyl, and
R⁴ is a saturated C₆₋₉ linear, branched or cyclic hydrocarbon radical or a radical of formula (II)

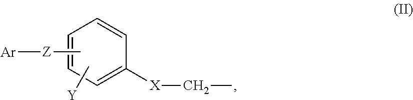

wherein

### X is S or O,

Y is a hydrogen atom or up to 2 halogen atoms,
Z is a single bond or a divalent radical being O, S, —CR₂—, in which R is hydrogen or C₁₋₄ alkyl, or other divalent radical with 2-10 carbon atoms and, optionally, O and/or S atoms linked in the form of a chain, wherein—if the radicals contain 2 or more O and/or S atoms—the latter are separated from one another by at least 2 carbon atoms, and it also being possible for 2 adjacent carbon atoms to be linked together by a double bond, and the free valencies of the carbon atoms being saturated by a hydrogen atom and/or C₁₋₄ alkyl groups,
Ar is an aromatic ring system which has up to two rings and which may be substituted by up to three radicals from the group of fluorine, chlorine, bromine, methoxy, C₁₋₄ alkyl, trifluoromethyl and trifluoromethoxy, salts and/or solvates thereof,
for use in the treatment of a tumor bearing deregulated, preferably upregulated, MYC oncoproteins, wherein said compound is capable of increasing UTR-dependent expression of at least one MYC gene.

### BRIEF DESCRIPTION OF THE DRAWINGS

The invention will now be described, by way of example only, with reference to the enclosed figures of drawing, wherein:

FIG. 1: Outline of the restriction site-free cloning method.

FIG. 2: Counter-screening with 112 compounds in CHP134-MYCN#3 and CHP134-CTRL#19 neuroblastoma cell clones, to discard those hits which produced luciferase reported upregulation because of action on the reporter plasmid promoter. Luciferase raw values were normalized to the values from the corresponding untreated cells. The data are represented as the means±standard deviation of triplicate experiments.

FIG. 3: Effect of 2 μM CPX treatment for 24 h on CHP134 neuroblastoma stable clones. Data are reported as means±standard deviation of triplicate experiments. Numbers over the bars indicate the fold increase in luminescence units upon CPX treatment for each clone.

FIG. 4: WST-1 assay with 112 compounds at the concentration of 2 μM in the CHP134-MYCN#3 neuroblastoma cell clone. Analysis was performed 24 h and 48 h after treatment. Data are reported as means±standard deviation of triplicate experiments.

FIG. 5: Effect of increasing concentrations of CPX on seven neuroblastoma cell lines. Cells were treated with CPX at the defined concentrations for 48 h followed by viability determination by the WST-1 assay. Data are reported as mean percentage of growth±standard deviation.

FIG. 6: CPX induces cell death and apoptosis in CHP134 neuroblastoma cells. Cells were treated for 24 and 48 h. with defined concentrations of CPX and then stained with Propidium Iodide (PI) and FITC-Annexin V. Numbers indicate the percentage of apoptotic/dead cells (P1) and pre-apoptotic cells (P2) respectively.

FIG. 7: Dose-response curve showing the cvtotoxic effect of Ciclopirox olamine and Piroctone olamine on CHP134 and SiMa neuroblastoma cell line after 48 hours. Points represent the treatment (0.33-1-3.3-10-33-100 μM), as the average of three technical replicates±SD

FIG. 8: Immunofluorescence analysis of MYCN expression on CHP134 neuroblastoma cells treated with CPX at different concentrations for 24 h.

### DETAILED DESCRIPTION OF THE INVENTION

In the following description, numerous specific details are given to provide a thorough understanding of embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The headings provided herein are for convenience only and do not interpret the scope or meaning of the embodiments.

The “oncogene addiction” concept of cancer therapy relies on the fact that cancer cells become dependent on activated oncogenes, and therefore suppression of oncogene expression or activity can selectively impair cancer cell survival.

The present inventors provide here proof of the opposite concept of cancer therapy by “oncogene overdose”, by which an activated oncogene in a tumor is vice versa further upregulated to provide a therapeutic effect. This paradoxical strategy was surprisingly revealed in its possibility to the inventors after the results of a screening conducted using a reporter gene assay for compounds able to modulate MYCN oncogene expression in MYCN-amplified neuroblastoma cells acting through its 3′UTR.

Despite the fact that the rationale of the screening was to identify compounds able to downregulate MYCN expression so reducing the oncogenic potential of MYCN and induce cytotoxicity, the present inventors noted that 4 compounds were also selected in the screening as able to upregulate MYCN, but inducing efficient cytotoxicity as well. Among these 4 molecules, 3 were well-known chemotherapeutic agents belonging to the anthracycline class, which is used in the first line treatment of neuroblastomas (doxorubicin, daunorubicin and epirubicin), therefore providing confirmation of the validity of the approach. The fourth molecule was an antifungine compound topically used in treating micoses, ciclopirox olamine.

A possible reason of this paradoxical outcome could rely on the fact that in several instances oncogene activation elicits by itself phylogenetically conserved programs of tumor suppression (by cell death, differentiation or senescence) which are possibly an intrinsic safeguard mechanism active in stem cells to prevent excess, uncontrolled proliferation.

Clonal selection during the slow process of tumor development (especially when a step-by-step, gradual event of oncogene activation as gene amplification is one of the major driving forces of clone aggressiveness) raises barriers to these programs, under the form of a variety of mutations inactivating them.

Therefore, an acute, strong additional over-activation of the already activated oncogene could determine a massive overcoming of these barriers, leading to extinction of the cell clones bearing the activated oncogene.

While specific upregulation of oncogene protein activity or of oncogene transcription would be difficult to be achieved pharmacologically, an increase in oncogene protein content, as well as a decrease, is intrinsically obtained by screening for activities perturbing post-transcriptional gene expression controls, specifically on mRNA stability and/or translational efficiency.

A preferred embodiment of the present disclosure concerns compounds of formula (I):

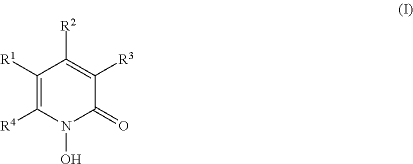

wherein
R¹, R² and R³, which are identical or different, are hydrogen atom or C₁₋₄ alkyl, and
R⁴ is a saturated C₆₋₉ linear, branched or cyclic hydrocarbon radical or a radical of formula (II)

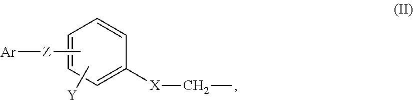

wherein

### X is S or O,

Y is a hydrogen atom or up to 2 halogen atoms,
Z is a single bond or a divalent radical being O, S, —CR₂—, in which R is hydrogen or C₁₋₄ alkyl, or other divalent radical with 2-10 carbon atoms and, optionally, O and/or S atoms linked in the form of a chain, wherein—if the radicals contain 2 or more O and/or S atoms—the latter are separated from one another by at least 2 carbon atoms, and it also being possible for 2 adjacent carbon atoms to be linked together by a double bond, and the free valencies of the carbon atoms being saturated by a hydrogen atom and/or C₁₋₄ alkyl groups,
Ar is an aromatic ring system which has up to two rings and which may be substituted by up to three radicals from the group of fluorine, chlorine, bromine, methoxy, C₁₋₄ alkyl, trifluoromethyl and trifluoromethoxy, salts and/or solvates thereof,
for use in the treatment of a tumor bearing deregulated, preferably upregulated, MYC oncoproteins, wherein said compound is capable of increasing UTR-dependent expression of at least one MYC gene.

Preferred compounds are 1-hydroxy-2-pyridinone (claim 2), ciclopirox (claim 3), ciclopirox olamine (claim 4), piroctone (claim 5), piroctone olamine (claim 6), and rilopirox (claim 7).

Further preferred compounds are 1-hydroxypyridine-2-thione (omadine) and 3-hydroxy-1,2-dimethylpyridin-4-one (deferiprone).

These compounds are endowed with very low systemic toxicity and with much higher bioavailability in comparison with conventional antitumor agents. For example, the acute lethal dose 50 (LD50) for ciclopirox olamine and for deferiprone in the rat is respectively 2350 mg/kg and 2000 mg/kg, while the same value for doxorubicin and cisplatin is respectively 10.5 mg/kg and 25 mg/kg. The plasma concentration of deferiprone in humans for iron overload treatment in transfused thalassemia patients is 250 μM and over, while that for high dose infusional doxorubicin in tumor bearing patients is 0.1 μM. Therefore, the systemic toxicity between this class of compounds and a conventional antitumor class of compounds as anthracyclines could be as much as 200-fold less, while their plasma concentration, and presumably their bioavailability, at doses used in therapy could be even more than 2500-fold higher.

The compounds referenced above have been identified by means of method of screening for a compound for treatment of a tumor having at least one activated oncogene, wherein the compound is capable of increasing the expression of the at least one activated oncogene through direct or indirect action on the untranslated regions of the mRNAs transcribed from the oncogene locus of interest, the method comprising:

i) contacting a compound with a cell comprising a nucleic acid construct, wherein the nucleic acid construct comprises a reporter gene coding sequence flanking or linked to the at least one oncogene untranslated region sequence or fragments thereof; and

ii) detecting expression of a reporter polypeptide encoded by the reporter gene coding sequence; wherein an increase in the level of expression of the reporter polypeptide in the presence of the compound relative to the level of expression of the reporter polypeptide in absence of the compound indicates that the compound increases the expression of the at least one activated oncogene through direct or indirect action on the untranslated regions of the mRNAs transcribed from the oncogene locus of interest.

Preferably, the untranslated region consists of the 3′ and/or the 5′ untranslated regions and/or segments or combinations of segments thereof.

The cell used in the screening method is, preferably a cancer cell, more preferably a human cancer cell. For the screening of compounds active against neuroblastoma the cells may be selected among the cell lines: CHP134, KELLY, CHP212, CHP134, MHH-NB-11, STA-NB-1, STA-NB-7, LA-N-1, SK-N-BE(2), SIMA, LA-N-2, SK-N-DZ, IMR32, SIMA, CHP126 (bearing MYCN amplification at different MYCN copy number) or, as comparison, SK-N-AS, SK-N-MC, SK-N-SH, SK-N-FI, NB69 (not bearing MYCN amplification).

The reporter gene is preferably selected among luciferase, green fluorescent protein, red fluorescent protein, yellow fluorescent protein, β-galactosidase, β-glucoronidase, β-lactamase, secreted placental alkaline phosphatase.

The tumor having at least one activated oncogene can be selected among neuroblastoma, medulloblastoma, retinoblastoma, small cell lung carcinoma, glioma, alveolar rhabdomyosarcoma, primitive neuroectodermal tumor, breast cancer, esophageal cancer, cervical cancer, ovarian cancer, head and neck cancer.

By disclosing the invention, the present inventors refer to MYCN protein only as an example, being the concept extended to the whole MYC gene family.

The compound capable of increasing the expression of activated oncogenes through direct or indirect action on the untranslated regions of the mRNAs transcribed from their loci may be selected among small molecule compound and macromolecule compound; preferably the small molecule compounds may be selected among: chemical small molecule compounds, normal and chemically modified antisense and sense RNAs, normal and chemical modified antisense DNA and RNA oligonucleotides, normal and chemically modified DNA and RNA decoy oligonucleotides, normal and modified DNA and RNA antagomirs (microRNA antagonists), normal and chemically modified RNA oligonucleotides acting as microRNA “sponges”, wherein said compound acts on cis-acting sequences present in the 5′ untranslated region or in the 3′ untranslated region of the at least one activated oncogene.

By disclosing the invention, the present inventors refer to MYCN protein only as an example, being the concept extended to the whole MYC gene family.

MYC family proteins (MYC, MYCN, and MYCL) are a paradigm for this mechanism of screening, since their activation elicits a well documented, powerful “inwired” tumor suppression program, acting through cell death, cell differentiation or cell senescence (PMID:20382143).

Specifically, MYCN results to be essential for maintaining a population of proliferating undifferentiated progenitor cells in the developing brain (PMID: 12381668), but at the same time it initiates the migration and the neuronal differentiation of neural crest cells in the sympathetic ganglia (PMID: 9169842), and it is endowed with pro-apoptotic properties at least in specific settings, such as TRAIL-induced triggering of the cell death machinery [PMID: 15632181], and drug-induced cell damage [PMID: 11107122, PMID: 17141950]. So, together with the unrestricted proliferation program involved in tumorigenesis at least two “rescue” counteracting, tumor suppressor programs, differentiation and apoptosis, are elicited by MYCN.

The present inventors have performed a detailed computational study of the 3′UTR of the MYCN protein, and found that it is almost entirely highly conserved in vertebrate phylogenesis, it is bound from experimental evidence by 2 RNA binding proteins and from bioinformatic prediction it bears potential binding sites for at least other 5 RNA binding proteins; it also bears binding sites for at least 17 microRNAs. All of this would predict for a highly regulated 3′UTR. Moreover, microarray-based mRNA profiling of 14 MYCN-amplified parental neuroblastoma cell lines (cell lines directly derived from the original tumor and not by in vitro cell subcloning) show different profiles of correlation between protein (detected by western blotting) polysomal mRNA, cellular mRNA levels (detected by quantitative RT-PCR) and degree of MYCN gene amplification (detected by array CGH analysis). This indirectly shows that during the process of MYCN amplification in the neuroblastoma cell lineage post-transcriptional controls act differently in different tumors, possibly due to alterations in these controls.

The above reported observations are in favor of a MYCN gene expression modulability by interferences exerted at the level of the 3′UTR of MYCN mRNA. Bioinformatic annotation of the MYC and MYCL 3′UTRs also showed a richness of potentially functionally cis element, of which some are demonstrated in the scientific literature, suggesting that these two genes also could be exogenously post-transcriptionally controlled acting on the 3′UTR.

The present inventors designed at the origin an high-throughput screening model aimed at finding MYCN downregulating small compounds expected to produce a specific cytotoxity effect on MYCN amplified neuroblastoma cells, being this lesion the main negative prognostic determinant of high risk neuroblastoma patients. The present inventors unexpectedly also found MYCN upregulating small compounds which nonetheless produced a very efficient cytotoxicity effect on MYCN amplified neuroblastoma cells.

By the high-throughput screening for compounds acting on the 3′UTR of MYCN mRNA, the present inventors have specifically identified ciclopirox olamine (CPX), a well known antimycotic drug, as a determinant of MYCN posttranscriptional upregulation and MYCN-induced cell death in neuroblastoma cells.

### Example 1

### Generation of pcDNA5/FRT-MYCN Plasmid

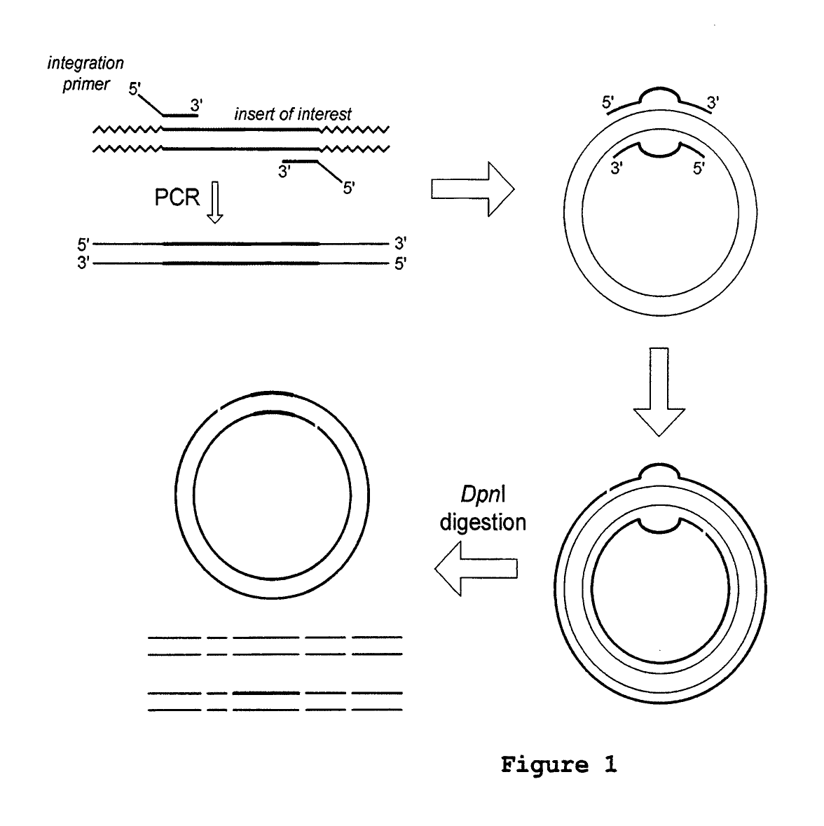

MYCN 3′UTR was inserted initially into the pcDNA5/FRT plasmid (Invitrogen) by the restriction site-free cloning method outlined in FIG. 1 (Cheng et al., 2000).

Briefly, two DNA integration primers were designed so that their nucleotide sequences were homologous to the sequence of MYCN 3′UTR (NG_{—}007457.1) at the 3′ portion and to the insertion region sequence of the pcDNA5/FRT vector in the 5′ portion.

The primer sequences are:

(SEQ ID No.: 1)
5′-GCCAAGAAGGGCGGCAAGATCGCCGTGTAAACGCTTCTCAAAACTGGACAGTCAC-3′
for the forward primer,
and
(SEQ ID No.: 2)
5′-CTTAATGCGCCGCTACAGGGCGCGTGGCCCCCCAACCAGGATTGTACAG-3′
for the reverse primer.

MYCN 3′UTR was first amplified in PCR by Platinum Pfx DNA polymerase (Invitrogen) with the forward and reverse integration primers (SEQ ID No.: 1 and 2) and gDNA from CHP134 cells (ECACC) as a template. A 30 cycle PCR program was applied, with denaturing at 94° C. for 15 s, annealing at 64° C. for 30 s and extension at 68° C. for 2 min with a final step of extension at 68° C. for 10 min. The product of this PCR was separated on a 1% agarose gel in TBE buffer, purified using the QIAquick gel extraction kit (Qiagen) and quantified by Nanodrop.

Next, the PCR product was extended by DNA polymerase using the pcDNA5/FRT vector as a template.

A 50 μl thermal cycling elongation reaction consisted of 50 ng pcDNA5/FRT, 200 ng purified PCR product, 200 μM dNTPs each and 2.5 U PfuUltra High-Fidelity polymerase (Stratagene) in its 1×PCR buffer. The thermal cycle program was denaturation at 95° C. for 30 sec, annealing at 55° C. for 1 min and elongation at 68° C. for 15 min with 35 cycles.

After the reaction, 10 U of restriction enzyme DpnI was added to 9 μl of PCR to digest for 2 h at 37° C. The designed plasmid is selected after DpnI digestion as DpnI cuts the parental and hybrid plasmids.

3 μl of DpnI-treated PCR mixture was taken to transform Top10 chemically competent E. coli cells (Invitrogen) according to the protocol of the supplier. E. coli colonies were checked for the presence of MYCN 3′UTR by PCR screening. A typical 12 μl PCR mixture consisted of 0.2 mM forward primer 5′-CGCAAGATCCGCGAGATTC-3′(SEQ ID No.:3), 0.2 mM reverse primer 5′-GCAAGTGTAGCGGTCACG-3′ (SEQ ID No.:4), 0.2 mM dNTPs each, 1.5 mM MgCl₂ and 0.5 U Platinum Taq DNA polymerase in its 1×PCR buffer. The initial denaturation step at 94° C. for 5 minutes was followed by 35 cycles of PCR amplification as follows: 94° C. for 30 seconds, 58° C. for 30 seconds, 72° C. for 2 minutes with a final step of extension at 72° C. for 5 minutes. The PCR products were visualized by agarose gel electrophoresis.

pcDNA5/FRT-MYCN plasmid was prepared from 250 ml overnight culture of transformed E. coli using Qiagen EndoFree plasmid maxi kit according to the manual instruction of the supplier.

### Example 2

### Generation of pGL4.26-MYCN3UTR and -CTRL Plasmids

For stable transfection two vectors—pGL4.26-MYCN3UTR and pGL4.26-CTRL carrying hygromycin B resistance gene—were generated.

To obtain pGL4.26-MYCN plasmid, two oligonucleotides 5′-CTAGAAAGTATAATCGATAAG-3′ (SEQ ID No.:5) and 5′-GATCCTTATCGATTATACTTT-3′ (SEQ ID No.:6) were designed so that by annealing a double stranded oligonucleotide was obtained with 5′- and 3′-protruding ends, representing XbaI and BamHI restriction half sites.

The pGL4.26 vector (Promega) was digested with NheI and BamHI enzymes to remove luc2 gene together with SV40 late polyA signal resulting in the pGL4.26 backbone with 5′- and 3′-protruding ends, representing NheI and BamHI restriction half sites (designated as pGL4.26×(MheI/BamHI)). The CMV promoter together with luc2 gene followed by MYCN 3′UTR was cut out with SpeI and XbaI restrictases from the MYCN-pcDNA5/FRT construct described above. A subsequent ligation of the pGL4.26×(NheI/BamHI) backbone, the CMV promoter-luc2 gene-MYCN 3′UTR×(SpeI/XbaI) fragment and the adaptor oligonucleotide described above yielded the desired pGL4.26-MYCN3UTR expression vector.

The used pGL4.26-CTRL vector represented a pGL4.26 plasmid with an inserted CMV promoter. pGL4.26-CTRL plasmid resulted from a ligation reaction between pGL4.26×(KpnI/BsrGI) backbone and a fragment containing CMV promoter. The latter was isolated from pGL4.26-MYCN3UTR by digestion with KpnI and BsrGI restrictases. In this way, pGL4.26-MYCN3UTR and pGL4.26-CTRL plasmids differed from each other exclusively in the region following luc2 gene.

### Example 3

### Stable Transfection of CHP134 Cells

CHP134 cells were grown as adherent monolayers at 37° C., 5% CO₂/air in RPMI 1640 (Lonza) supplemented with 10% fetal bovine serum (Lonza) and 10 mM L-glutamine (Lonza). CHP134 cells were transfected according to the following protocol: 100 μl of OPTI-MEM (Gibco), 2 μg of pGL4.26-MYCN or —CTRL plasmid and 6 μl of the TurboFectin 8.0 (OriGene) were mixed in a tube. After 30 min incubation at room temperature, the mixture was added dropwise to the CHP134 cells growing on 12-well plates in complete RPMI 1640. In 5 h media was changed for RPMI 1640 with 20% FBS.

On the next day the cells were trypsinized and transferred to a 100-mm dish. After 2 days selection of stably transfected cells was started by adding hygromycin B (Invitrogen) to the medium to obtain a final concentration of 110 μg/ml. The media with hygromycin B was changed every 3-4 days.

Approximately 18 days after transfection clones were transferred to a 12-well plate by picking with a plastic tip. When enough cells for a specific clone were available, the major part of the cells were collected, cryopreserved and stored in liquid nitrogen; the remaining cells were collected in microcentrifuge tubes at the concentration (2-5)×10⁶ cells per tube and stored at −80° C. as pellets.

Clones were selected based on moderate luciferase activity and intact CMV promoter and MYCN 3′UTR or SV40 late polyA regions.

The luciferase activity was estimated using Luciferase assay (Promega) according to the protocol of the supplier with slight modifications. Briefly, the clones' pellets were thawed on ice and lysed in 100 μl of 1× passive lysis buffer followed by three freeze-thaw cycles to ensure complete lysis. The lysates were centrifuged for 20 min at the highest speed at 4° C. The supernatants were transferred into fresh tubes. To the white flat-bottom 96-well plate, containing 20 μl of cell lysate per well, 100 μl of Luciferase Assay reagent was added per well. The light produced was measured immediately with the Tecan F200 multiplate reader (Tecan). 5 μl of the same lysates were used to measure protein quantity by Bradfrod assay. Finally, luminescent units normalized to protein amount were inter-compared. An integrity of CMV promoter and MYCN 3′UTR was verified by PCR. The pelleted cells from clones stored at −80° C. were thawed on ice, gDNA was purified with DNeasy Blood & Tissue kit (Qiagen) and quantified.

CMV promoter was amplified in a nested PCR. A typical 25 μl first PCR mixture consisted of 100 ng gDNA, 0.2 mM forward primer 5′-CTAGCAAAATAGGCTGTCCCCAGTG-3′(SEQ ID No.:7), 0.2 mM reverse primer 5′-CACACCACGATCCGATGGTTTG-3′ (SEQ ID No.:8), 0.2 mM dNTPs each, 1.5 mM MgCl₂ and 2.5 U Platinum Taq DNA polymerase in its 1×PCR buffer. The initial denaturation step at 94° C. for 2 minutes was followed by 35 cycles of PCR amplification as follows: 94° C. for 30 seconds, 63° C. for 30 seconds, 72° C. for 2 minutes with a final step of extension at 72° C. for 5 minutes.

In the second PCR 1 μl of the respective 1.PCR mixture (1:10 dilution) served as a template. The second PCR mixture was equivalent to the first one apart from primers which were the following: forward 5′-CGTTACATAACTTACGGTAAATGG-3′ (SEQ ID No.:9) and reverse 5′-GAAGTACTCGGCGTAGGTAATG-3′ (SEQ ID No.:10) primers. The second PCR was carried out using the following thermal profile: initial denaturation at 94° C. for 2 min followed by 35 cycles of denaturation at 94° C. for 30 sec, annealing at 57° C. for 30 sec, elongation at 72° C. for 2 minutes with a final step of extension at 72° C. for 5 minutes. The PCR products were visualized by agarose gel electrophoresis.

MYCN 3′UTR was amplified in nested PCR. A typical 25 μl first PCR mixture consisted of 100 ng gDNA, 0.2 mM forward primer (SEQ ID No.:3), 0.2 mM reverse primer (SEQ ID No.:4), 0.2 mM dNTPs each, 1.5 mM MgCl₂ and 2.5 U Platinum Tag DNA polymerase in its 1×PCR buffer. The initial denaturation step at 94° C. for 2 minutes was followed by 35 cycles of PCR amplification as follows: denaturation at 94° C. for 30 seconds, annealing at 58° C. for 30 seconds, elongation at 72° C. for 2 minutes with a final step of extension at 72° C. for 5 minutes.

In the second PCR 1 μl of the respective first PCR mixture (1:10 dilution) served as a template. The second PCR mixture was equivalent to the first one apart from the primers which were the following: forward (SEQ ID No.:1) and reverse (SEQ ID No.:2) primers. A 35 cycle, two-step PCR program was applied, with denaturing at 94° C. for 30 seconds and annealing/extension at 72° C. for 2 minutes with a final step of extension at 72° C. for 5 minutes. The PCR products were visualized by agarose gel electrophoresis.

### Example 4

### An High Throughput Screening for MYCN 3′UTR-Increased Translation Compounds

Reporter constructs containing the firefly luciferase reporter gene under the control of the CMV viral promoter and followed by either the whole MYCN 3′UTR (pGL4.26-MYCN3UTR) or the only SV40 poly(A) region (pGL4.26-CTRL) as a control were produced as disclosed above.

The CHP134 neuroblastoma cell line was used to generate stable transfection clones as disclosed in Example 3.

The screening was carried out in the CHP134-MYCN#3 stable clone with the Spectrum Collection small molecule library (MicroSource Discovery) composed of 2000 compounds stored at 10 mM in DMSO: 800 drugs that have been introduced in the US, 200 drugs that are limited in use to Europe and Japan, 580 natural products, 420 compounds with reported biological activities.

The assay was run in triplicates in 96-well plates. CHP134-MYCN stable clone cells were trypsinized, harvested and resuspended in culture medium. Tecan Multichannel arm (MCA96) of a Tecan EVO 200 robot (Tecan) was used to add 150 uL containing 15000 cells to the 96-well white CulturePlate-96 (Perkin Elmer).

After adhesion, the 10 mM compounds were diluted to 75 uM in PBS and immediately pipetted into the 96 wells in order to have a final concentration of 2 uM in the cells. Baseline controls were obtained treating the first column wells with PBS+DMSO at the same final concentration of the samples.

Luciferase activity was assessed using OneGlow Luciferase assay (Promega) according to the manufacturer's method, after 24 h of incubation at 37° with 5% CO₂ and 100% relative humidity. Luminescence signal was read on a Tecan F200 multiplate reader (Tecan) integrated with the robot.

Hits were selected from the primary screening using a robust Z score method. Z score normalizing method is calculated as:

Z=(xᵢ−median)/MAD eq. (1)

where xᵢ is the raw measurement on the i^{th} compound, median and MAD are the median and median absolute deviation, respectively.

The RankProduct method was applied between three replicates of all plates in order to detect hits by pfp (threshold set to 0.1). This gave 59 hits which induced reporter over-expression, and 80 down-regulated hits.

The majority of the hits (Table 1) were then checked for reproducibility and cytotoxic activity, the most interesting compounds were also tested for dose-responsiveness. The same hits were subsequently subjected to counter-screening with the stable clone CHP134-CTRL#19 expressing the control pGL4.26-CTRL plasmid, to segregate plasmid promoter-dependent transcriptional control effects.

TABLE 2

| | |
|---|---|
| ID | COMPOUND NAME |

PLATE 1

| | | | |
|---|---|---|---|
| 1 | 01_B08 | 00330001 | DACTINOMYCIN |
| 2 | 01_B09 | 00330002 | MITOMYCIN C |

PLATE 2

| | | | |
|---|---|---|---|
| 3 | 02_D03 | 01500189 | CICLOPIROX OLAMINE |
| 4 | 02_E03 | 01500205 | COLCHICINE |
| 5 | 02_F07 | 01500223 | DAUNORUBICIN |
| 6 | 02_H05 | 01500246 | DIGITOXIN |
| 7 | 02_H06 | 01500247 | DIGOXIN |

PLATE 4

| | | | |
|---|---|---|---|
| 8 | 04_A08 | 01500375 | MECHLORETHAMINE |
| 9 | 04_B05 | 01500387 | MERCAPTOPURINE |
| 10 | 04_B06 | 01500388 | MESTRANOL |
| 11 | 04_C03 | 01500398 | METHOTREXATE(+/−) |
| 12 | 04_H07 | 01500473 | PHENAZOPYRIDINE HYDROCHLORIDE |

PLATE 5

| | | | |
|---|---|---|---|
| 13 | 05_D07 | 01500521 | PYRVINIUM PAMOATE |
| 14 | 05_H06 | 01500573 | THIOGUANINE |

PLATE 6

| | | | |
|---|---|---|---|
| 15 | 06_C09 | 01500611 | VINBLASTINE SULFATE |
| 16 | 06_D04 | 01500618 | ACRIFLAVINIUM HYDROCHLORIDE |
| 17 | 06_D09 | 01500644 | PHENYLMERCURIC ACETATE |
| 18 | 06_E11 | 01500674 | MYCOPHENOLIC ACID |
| 19 | 06_F03 | 01500676 | OUABAIN |

PLATE 7

| | | | |
|---|---|---|---|
| 20 | 07_A02 | 01500873 | PIPERINE |
| 21 | 07_A03 | 01500903 | ETOPOSIDE |
| 22 | 07_B07 | 01501016 | FENBENDAZOLE |
| 23 | 07_C03 | 01501110 | MEBENDAZOLE |

PLATE 8

| | | | |
|---|---|---|---|
| 24 | 08_B11 | 01502198 | ANISINDIONE |
| 25 | 08_D05 | 01503059 | FLOXURIDINE |
| 26 | 08_H11 | 01503256 | AMSACRINE |

PLATE 9

| | | | |
|---|---|---|---|
| 27 | 09_A10 | 01503278 | MITOXANTHRONE HYDROCHLORIDE |
| 28 | 09_C10 | 01503650 | NABUMETONE |
| 29 | 09_E03 | 01503908 | PACLITAXEL |
| 30 | 09_G06 | 01504094 | TENIPOSIDE |
| 31 | 09_H08 | 01504179 | FEXOFENADINE HYDROCHLORIDE |

PLATE 11

| | | | |
|---|---|---|---|
| 32 | 11_C08 | 01505414 | BROMINDIONE |
| 33 | 11_F02 | 01505483 | DOXORUBICIN |
| 34 | 11_H06 | 01505672 | VINCRISTINE SULFATE |

PLATE 12

| | | | |
|---|---|---|---|
| 35 | 12_B05 | 01505708 | EPIRUBICIN HYDROCHLORIDE |

PLATE 13

| | | | |
|---|---|---|---|
| 36 | 13_A07 | 02300332 | PODOFILOX |
| 37 | 13_F02 | 01506084 | PROSCILLARIDIN |
| 38 | 13_H11 | 01501205 | LANATOSIDE C |

PLATE 14

| | | | |
|---|---|---|---|
| 39 | 14_A03 | 00100005 | ANTHOTHECOL |
| 40 | 14_A06 | 00100009 | CEDRELONE |
| 41 | 14_E04 | 00100291 | STROPHANTHIDIN |
| 42 | 14_H10 | 00100584 | GITOXIGENIN DIACETATE |

PLATE 15

| | | | |
|---|---|---|---|
| 43 | 15_B03 | 00100688 | DIGOXIGENIN |
| 44 | 15_B04 | 00100698 | CYMARIN |
| 45 | 15_B06 | 00100749 | STROPHANTHIDINIC ACID LACTONE ACETATE |
| 46 | 15_B07 | 00102007 | FORMONONETIN |
| 47 | 15_C03 | 00200007 | GAMBOGIC ACID |
| 48 | 15_C07 | 00200013 | ROTENONE |
| 49 | 15_C09 | 00200022 | AKLAVINE HYDROCHLORIDE |
| 50 | 15_F02 | 00200484 | DEOXYSAPPANONE B 7,4′-DIMETHYL ETHER |
| 51 | 15_G03 | 00200789 | DAIDZEIN |
| 52 | 15_G06 | 00200846 | APIGENIN |
| 53 | 15_G07 | 00200848 | DEOXYSAPPANONE B 7,3′-DIMETHYL ETHER |
| 54 | 15_H08 | 00201342 | DEOXYSAPPANONE B 7,3′-DIMETHYL ETHER ACETATE |

PLATE 16

| | | | |
|---|---|---|---|
| 55 | 16_A05 | 00201522 | GAMBOGIC ACID AMIDE |
| 56 | 16_A06 | 00201524 | DIHYDROGAMBOGIC ACID |
| 57 | 16_B03 | 00201604 | PYRROMYCIN |
| 58 | 16_B08 | 00201664 | CELASTROL |
| 59 | 16_E06 | 00210658 | DEHYDROVARIABILIN |
| 60 | 16_F07 | 00211126 | DIPHENYLUREA |
| 61 | 16_F08 | 00211249 | 7,4′-DIMETHOXYISOFLAVONE |
| 62 | 16_G03 | 00240645 | RETUSIN 7-METHYL ETHER |
| 63 | 16_G10 | 00240958 | 4′-METHOXYFLAVONE |

PLATE 17

| | | | |
|---|---|---|---|
| 64 | 17_A03 | 00300007 | EUPARIN |
| 65 | 17_F04 | 00310010 | HELENINE |
| 66 | 17_G09 | 00211950 | COSMOSIIN |
| 67 | 17_G11 | 00212097 | ONONETIN |
| 68 | 17_H07 | 00501332 | PHENACYLAMINE HYDROCHLORIDE |

PLATE 18

| | | | |
|---|---|---|---|
| 69 | 18_B02 | 01500709 | CHRYSIN |
| 70 | 18_B03 | 01500717 | 6,4′-DIHYDROXYFLAVONE |
| 71 | 18_B11 | 01500736 | 3,6-DIMETHOXYFLAVONE |
| 72 | 18_G06 | 01500986 | GITOXIN |
| 73 | 18_H07 | 01501197 | PRIMULETIN |
| 74 | 18_H10 | 01501207 | KINETIN RIBOSIDE |

PLATE 19

| | | | |
|---|---|---|---|
| 75 | 19_A04 | 01502223 | RESVERATROL |
| 76 | 19_A08 | 01502232 | CAMPTOTHECIN |
| 77 | 19_B07 | 01502247 | FISETIN |
| 78 | 19_D06 | 01503904 | PATULIN |
| 79 | 19_D07 | 01503906 | ANISOMYCIN |
| 80 | 19_E08 | 01503994 | CONVALLATOXIN |
| 81 | 19_E10 | 01504002 | BAICALEIN |
| 82 | 19_G06 | 01504041 | TRIACETYLRESVERATROL |
| 83 | 19_G07 | 01504044 | RESVERATROL 4′-METHYL ETHER |
| 84 | 19_G10 | 01504068 | DIOSMETIN |
| 85 | 19_H07 | 01504082 | DIHYDROCELASTROL |

PLATE 20

| | | | |
|---|---|---|---|
| 86 | 20_B05 | 01504411 | PICROPODOPHYLLOTOXIN ACETATE |
| 87 | 20_B06 | 01504412 | PODOPHYLLOTOXIN ACETATE |
| 88 | 20_C08 | 01505129 | PLUMBAGIN |
| 89 | 20_C11 | 01505135 | PIPLARTINE |
| 90 | 20_D05 | 01505142 | 2′,5′-DIHYDROXY-4-METHOXYCHALCONE |
| 91 | 20_D09 | 01505152 | 2′,4′-DIHYDROXY-4-METHOXYCHALCONE |
| 92 | 20_D10 | 01505153 | 2′,3-DIHYDROXY-4,4′,6′-TRIMETHOXYCHALCONE |
| 93 | 20_E06 | 01505177 | RUBESCENSIN A |
| 94 | 20_E07 | 01505182 | ISOFORMONONETIN |
| 95 | 20_G05 | 01505278 | 3-HYDROXY-3′,4′-DIMETHOXYFLAVONE |

PLATE 21

| | | | |
|---|---|---|---|
| 96 | 21_A04 | 01505490 | APIGENIN DIMETHYL ETHER |
| 97 | 21_C07 | 01600561 | LIQUIRITIGENIN DIMETHYL ETHER |
| 98 | 21_D05 | 10100003 | BIOCHANIN A |
| 99 | 21_E04 | 00201138 | DEGUELIN(−) |

PLATE 22

| | | | |
|---|---|---|---|
| 100 | 22_B11 | 00200833 | ACACETIN DIACETATE |
| 101 | 22_C07 | 01504123 | 10-HYDROXYCAMPTOTHECIN |
| 102 | 22_C10 | 01501113 | PERUVOSIDE |
| 103 | 22_G05 | 01503074 | ALEXIDINE HYDROCHLORIDE |

PLATE 23

| | | | |
|---|---|---|---|
| 104 | 23_A08 | 01504079 | TOMATINE |
| 105 | 23_E08 | 01505180 | 6,2′-DIMETHOXYFLAVONE |
| 106 | 23_F02 | 01505158 | 2,3-DICHLORO-5,8-DIHYDROXYNAPTHOQUINONE |
| 107 | 23_F07 | 01505328 | 4′-DEMETHYLEPIPODOPHYLLOTOXIN |

PLATE 24

| | | | |
|---|---|---|---|
| 108 | 24_C08 | 00300563 | TRICHLORMETHINE |
| 109 | 24_D02 | 01504410 | PICROPODOPHYLLOTOXIN |
| 110 | 24_E05 | 01502083 | N-(9-FLUORENYLMETHOXYCARBONYL)-L-LEUCINE |
| 111 | 24_G03 | 01505311 | DIBENZOYLMETHANE |
| 112 | 24_G11 | 01504101 | TETRACHLOROISOPHTHALONITRILE |

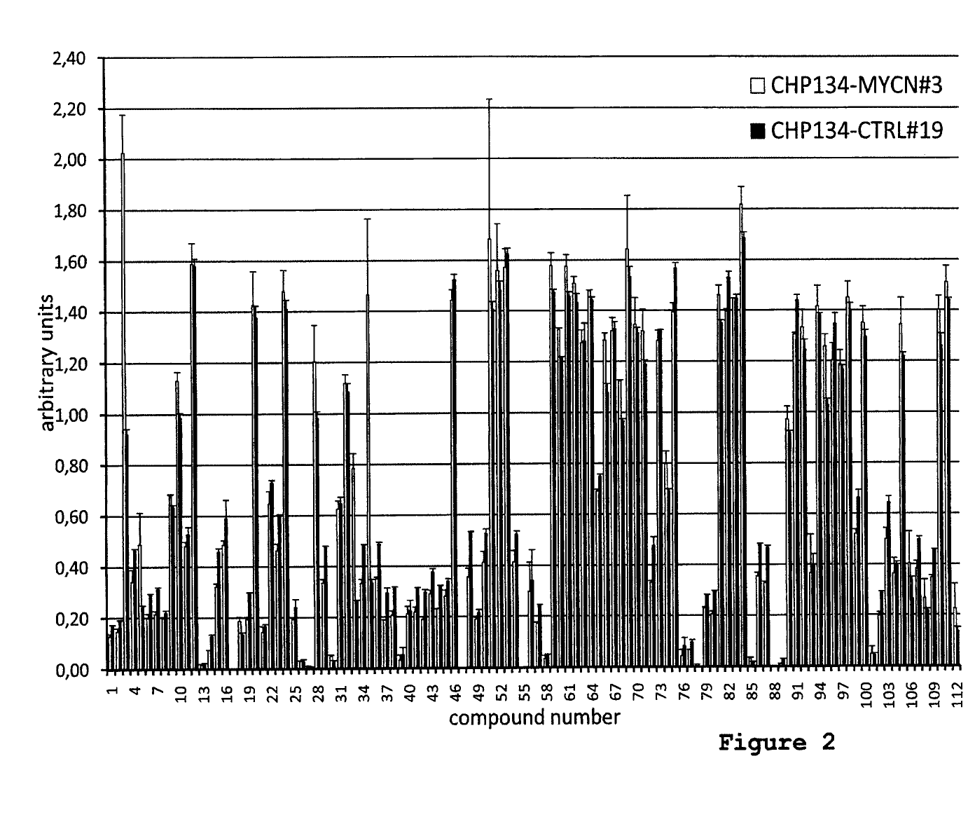

The counter-screening selected only 4 drugs as truly dependent on MYCN 3′UTR, three of which belonged to the anthracyclines class (doxorubicin, epirubicin, and daunorubicin), while the fourth was ciclopirox olamine (CPX), a synthetic antifungal compound belonging to the hydroxypyridones class (FIG. 2).

### Example 5

### Specificity of Ciclopirox Olamine (CPX) as a MYCN-Upregulating Drug in Neuroblastoma Cells

To verify the specificity of the CPX effect for the MYCN 3′UTR and its clone-independency, the treatment was repeated with the CHP134-MYCN#3 clone and the CHP134-CTRL#19 clones and with two independent others, again stably transfected with the pGL4.26-MYCN3UTR and the pGL4.26-CTRL plasmids (respectively, CHP134-MYCN#1 and the CHP134-CTRL#17 clones).

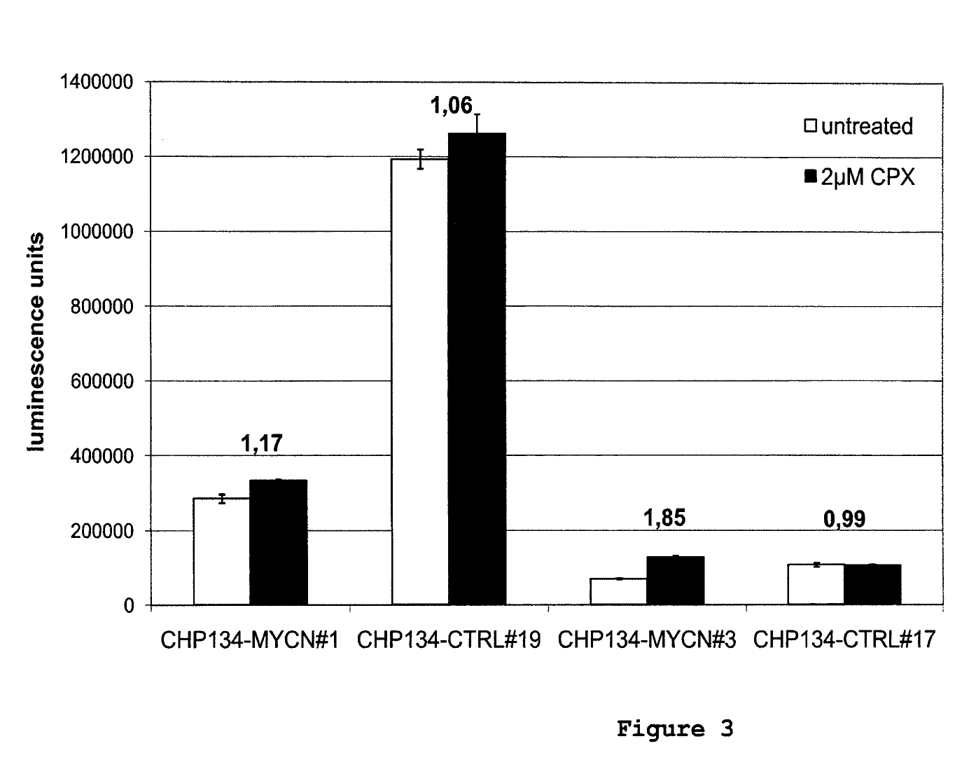

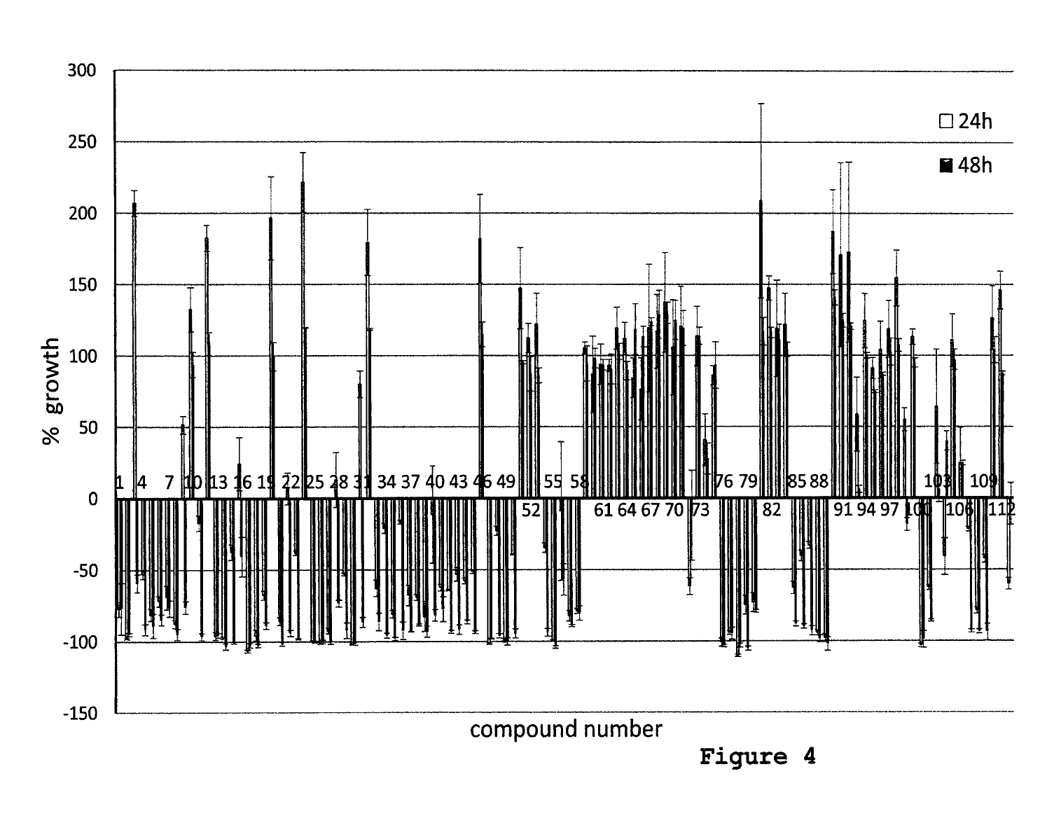

Treatment with CPX 2 uM and measurement of luciferase activity with the OneGlow luciferase assay after 24 h provided a confirmation of the screening results (FIG. 3). Therefore, it is possible to conclude that CPX elicited a clone-independent, MYCN 3′UTR specific effect of increased luciferase expression in CHP134 neuroblastoma cells.

### Example 6

### Cell Cytotoxicity of MYCN-Upregulating Drugs in Neuroblastoma Cells

a) Cell cytotoxicity was performed by WST1 assay (Roche) following the manufacturer's instruction: 15000 cells were plated in each well of 96-well transparent Spectra Plate (Perkin Elmer) in 150 ul of complete media.

After adhesion, cells were treated with the selected drugs (Table 1) at 2 uM concentration and incubated at 37° with 5% CO₂ and 100% relative humidity for 24 or 48 hours. At the defined endpoint, 15 uL of WST1 reagent were added and after 4 hours incubation at 37° with 5% CO₂ and 100% relative humidity, plates were read for absorbance at 450 nm in a Tecan F200 multiplate reader (Tecan).

Different measurements were needed in order to calculate the percentage growth: time zero (Tz: that represent a measurement of the cell population at the time of drug addition), control growth (C: measures the growth of the cells treated with vehicle only after 24 or 48 hours) and test growth (Ti: that represent a measurement of the cell population after 24 or 48 hours). Percentage growth is calculated as:

[(Ti−Tz)/(C−Tz)]×100 eq. (2)

for concentrations for which Ti>=Tz;

[(Ti−Tz)/Tz]×100 eq. (3)

for concentrations for which Ti<Tz.

The results of this experiment are shown in FIG. 4.

b) Cell cytotoxicity of CPX at different concentrations was performed by the WST1 assay (Roche) following the manufacturer's instruction. The dose-dependent effects of CPX on cell viability were tested in 7 neuroblastoma cell lines: CHP134 (ECACC), SK-N-BE(2) (ECACC), SIMA (DSMZ), LA-N-2 (ECACC), SK-N-MC (ECACC), SK-N-AS (ECACC), SK-N-SH (ECACC). Cells were plated in each well of 96-well transparent Spectra Plate (Perkin Elmer) in 150 ul at plating densities ranging from 5000 to 40000 cells/well depending on the doubling time of individual cell lines.

After adhesion, cells were treated with different concentration of CPX ranging from 66 nM to 66 uM concentration and incubated at 37° with 5% CO₂ and 100% relative humidity for 48 hours. At the defined endpoint, 15 ul of WST1 reagent were added and after 4 hours incubation at 37° with 5% CO₂ and 100% relative humidity, plates were read for absorbance at 450 nm in a Tecan F200 multiplate reader (Tecan).

Different measurements were needed in order to calculate the percentage growth: time zero (Tz: that represent a measurement of the cell population at the time of drug addition), control growth (C: measures the growth of the cells treated with vehicle only after 48 hours) and test growth (Ti: that represent a measurement of the cell population after 48 hours treatment with CPX).

Percentage growth is calculated as above by means of eq.s (2) and (3).

Growth inhibition of 50% (GI50) is calculated from:

[(Ti−Tz)/(C−Tz)]×100=50, eq. (4)

which is the drug concentration resulting in a 50% reduction of cells compared to the untreated control.

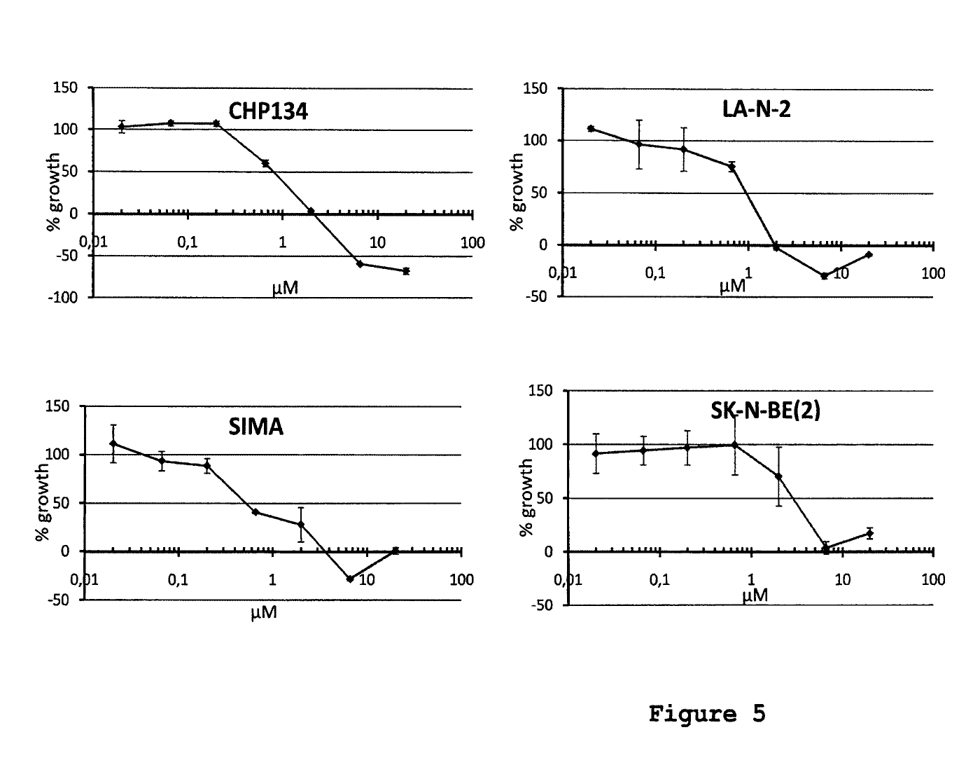

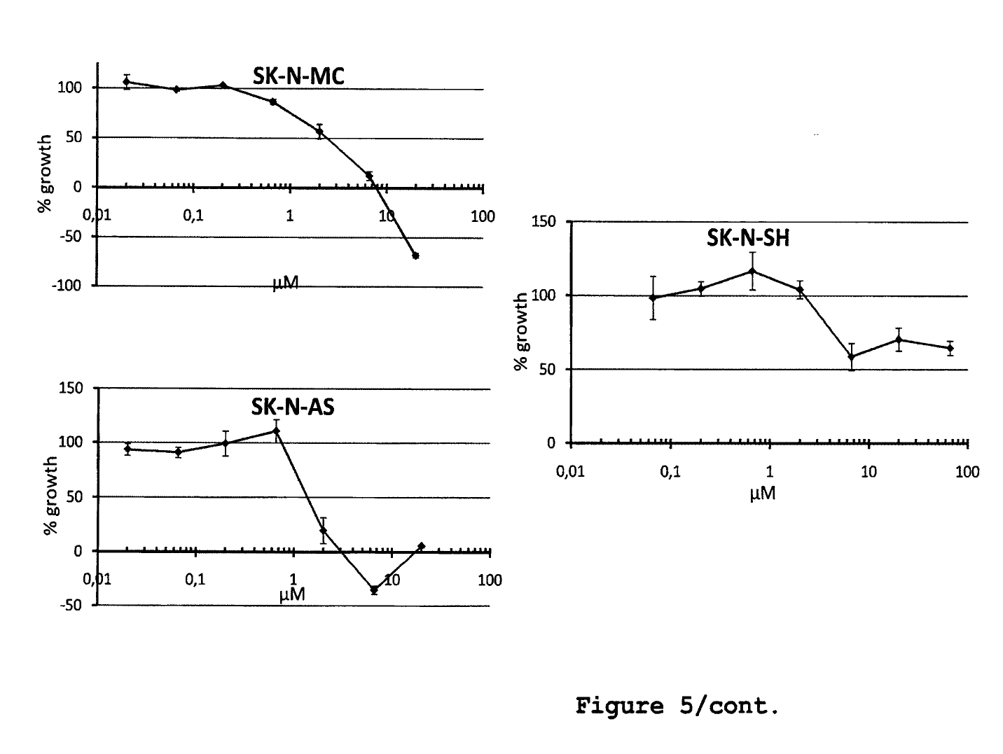

The results of this experiment are shown in FIG. 5.

c) Apoptosis assay was performed by flow cytometry. CHP134 cells were seeded 10 cm-dishes at the concentration 1×10⁶ cells under standard culture conditions. In three days cells were treated with CPX at different concentration for 24 and 48 hr. The cells were then harvested, washed with cold PBS and processed for apoptosis assay using the Annexin V-FITC Apoptosis Detection Kit I (BD Biosciences) by following the instructions of the manufacturer. Briefly, cells were stained with FITC-Annexin V and PI (Propidium Iodide) in order to allow for the identification of death cells (FITC and PI positive), viable cells (FITC and PI negative) and pre-apoptotic cells (FITC positive and PI negative). Flow cytometric analysis was performed with the BD FACS Canto (BD Biosciences).

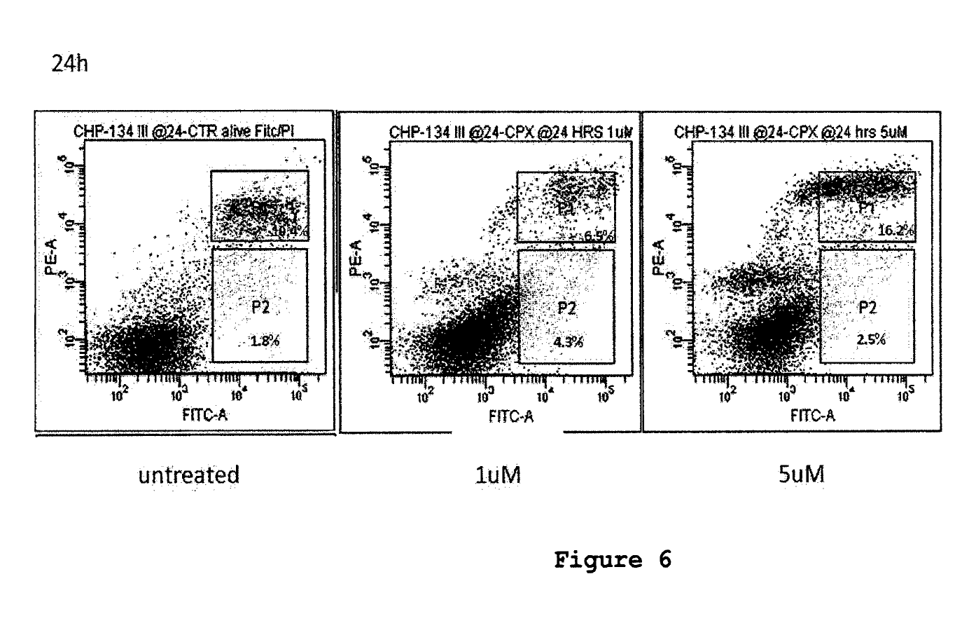

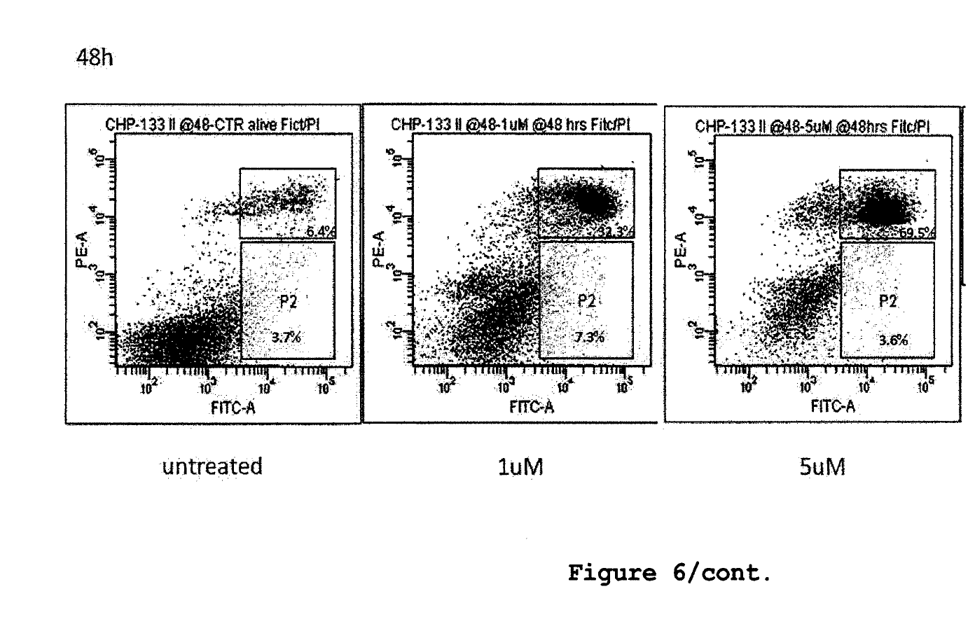

The results are shown, in FIG. 6. Numbers indicate the percentage of apoptotic/dead cells (P1) and pre-apoptotic cells (P2) respectively. A dose and time-dependent effect of CPX on cell viability is reflected by the increased percentage of apoptotic/dead cells (P1) and of pre-apoptotic+apoptotic/dead cells (P1+P2).

### Example 7

### Similarity of the Cytotoxic Profiles of CPX and Piroctone Olamine in Neuroblastoma Cells

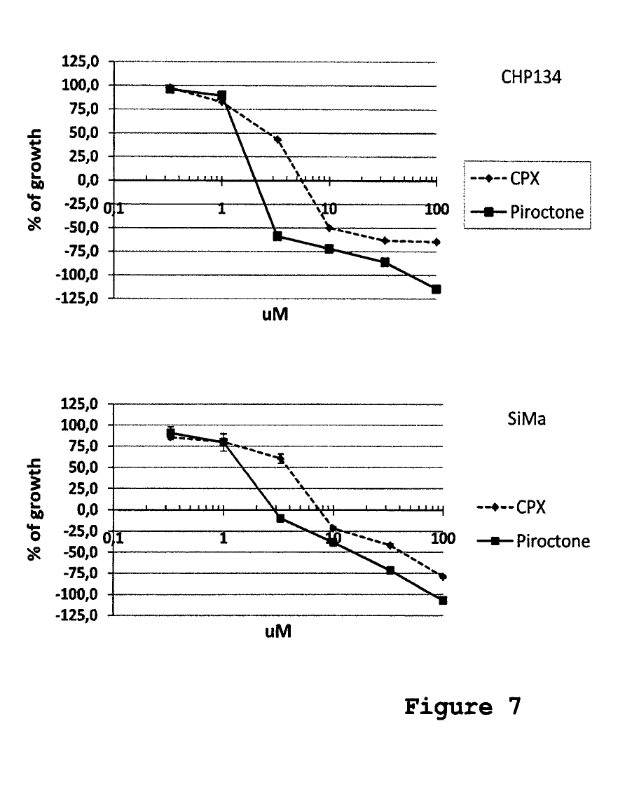

In order to understand if compounds of similar molecular structure to that of CPX could be also effective in inducing a cytotoxic activity, we treated two neuroblastoma cell lines, CHP134 and SiMa, with increasing concentrations of both compounds (0.33-1-3.3-10-33-100 μM), and we measured cytotoxicity after 48 hours as detailed before. The results, reported in FIG. 7 as the average plus standard error of three replicates, are in favor of an even stronger activity of piroctone olamine than CPX, but following the same profile. This suggests that the compounds claimed in this application of molecular structure similar to that of CPX can exert a comparable activity on neuroblastoma cells.

### Example 8

### CPX Determines an Increase in MYCN Protein Levels

To verify if the CPX enhancement effects on luciferase activity was accompanied by an increase in MYCN protein levels, an immunofluorescence analysis were performed on CHP134 cells plated in 96-well imaging plates (BD) and treated after adhesion for 24 h at different concentration of CPX. Cells were fixed by paraformaldehyde (PFA) 3.7%, permeabilized with 0.1% Triton X100, incubated with anti-MYCN mouse monoclonal antibody (ab16898, ABCAM) and stained with Alexafluor 488 rabbit anti-mouse secondary antibody.

Images were acquired by Operetta (Perkin Elmer) high content system and analyzed by the Harmony software.

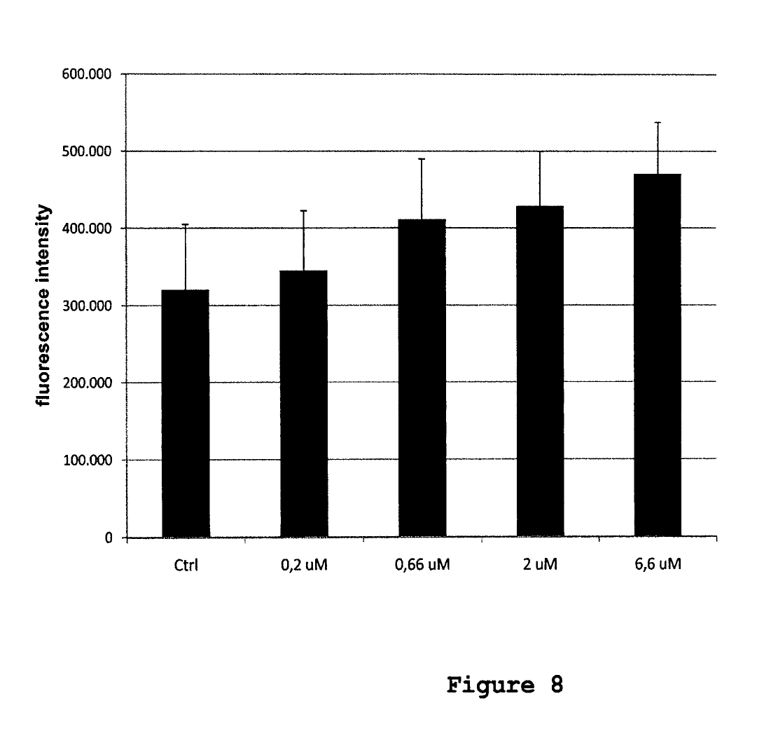

Results represent fluorescence intensity in the nuclear area previously identified by DAPI staining. It is clear a dose-dependent increase in MYCN nuclear immunostaining (FIG. 8).

Naturally, while the principle of the invention remains the same, the details of construction and the embodiments may widely vary with respect to what has been described and illustrated purely by way of example, without departing from the scope of the present invention.

### REFERENCES

G. J. Chen, N. Qiu, C. Karrer, P. Caspers and M. G. P. Page. Restriction Site-Free Insertion of PCR Products Directionally into Vectors. BioTechniques 28:498-505 (March 2000).
PMID:16510605—Tang X X, Zhao H, Kung B, Kim D Y, Hicks S L, Cohn S L, Cheung N K, Seeger R C, Evans A E, Ikegaki N. The MYCN enigma: significance of MYCN expression in neuroblastoma. Cancer Res. 2006 Mar. 1; 66(5):2826-33.
PMID:20382143—Larsson L G, Henriksson M A. The Yin and Yang functions of the Myc oncoprotein in cancer development and as targets for therapy. Exp Cell Res. 2010 May 1; 316(8):1429-37. Epub 2010 Apr. 9.
PMID:12381668—Knoepfler P S, Cheng P F, Eisenman R N. N-myc is essential during neurogenesis for the rapid expansion of progenitor cell populations and the inhibition of neuronal differentiation. Genes Dev. 2002 Oct. 15; 16(20):2699-712.
PMID:9169842—Wakamatsu Y, Watanabe Y, Nakamura H, Kondoh H. Regulation of the neural crest cell fate by N-myc: promotion of ventral migration and neuronal differentiation. Development. 1997 May; 124(10):1953-62.
PMID:15632181—Cui H, Li T, Ding H F. Linking of N-Myc to death receptor machinery in neuroblastoma cells. J Biol. Chem. 2005 Mar. 11; 280(10):9474-81.
PMID:11107122—Fulda S, Lutz W, Schwab M, Debatin K M. MycN sensitizes neuroblastoma cells for drug-triggered apoptosis. Med Pediatr Oncol. 2000 December; 35(6):582-4.
PMID:17141950—Paffhausen T, Schwab M, Westermann F Targeted MYCN expression affects cytotoxic potential of chemotherapeutic drugs in neuroblastoma cells. Cancer Lett. 2007 May 18; 250(1):17-24.

1. Compound of formula (I): 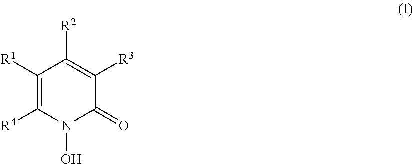
wherein
R¹, R² and R³, which are identical or different, are hydrogen atom or C₁₋₄ alkyl, and
R⁴ is a saturated C₆₋₉ linear, branched or cyclic hydrocarbon radical or a radical of formula (II) 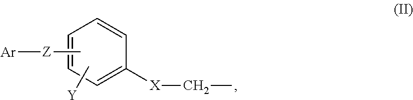
wherein
X is S or O,
Y is a hydrogen atom or up to 2 halogen atoms,
Z is a single bond or a divalent radical being O, S, —CR₂—, in which R is hydrogen or C₁₋₄ alkyl, or other divalent radical with 2-10 carbon atoms and, optionally, O and/or S atoms linked in the form of a chain, wherein—if the radicals contain 2 or more O and/or S atoms—the latter are separated from one another by at least 2 carbon atoms, and it also being possible for 2 adjacent carbon atoms to be linked together by a double bond, and the free valencies of the carbon atoms being saturated by a hydrogen atom and/or C₁₋₄ alkyl groups,
Ar is an aromatic ring system which has up to two rings and which may be substituted by up to three radicals from the group of fluorine, chlorine, bromine, methoxy, C₁₋₄ alkyl, trifluoromethyl and trifluoromethoxy, salts and/or solvates thereof,
for use in the treatment of a tumor bearing deregulated MYC oncoproteins, wherein said compound is capable of increasing UTR-dependent expression of at least one MYC gene.

2. Compound according to claim 1, wherein R¹, R², R³ and R⁴ are hydrogen atoms.

3. Compound according to claim 1, wherein R¹ is a methyl group, R² and R³ are hydrogen atoms, and R⁴ is cyclohexyl.

4. Compound according to claim 3, wherein the compound is a solvate with 2-aminoethanol.

5. Compound according to claim 1, wherein R¹ is a methyl group, R² and R³ are hydrogen atoms, and R⁴ is 2,4,4-trimethylpentyl.

6. Compound according to claim 5, wherein the compound is a solvate with 2-aminoethanol.

7. Compound according to claim 1, wherein R¹ is a methyl group, R² and R³ are hydrogen atoms, and R⁴ is 4-(4-chlorophenoxy)-phenoxy-methyl].

8. Compound 1-hydroxypyridine-2-thione for use in the treatment of a tumor bearing deregulated MYC oncoproteins, wherein said compound is capable of increasing UTR-dependent expression of at least one MYC gene.

9. Compound 3-hydroxy-1,2-dimethylpyridin-4-one for use in the treatment of a tumor bearing deregulated MYC oncoproteins, wherein said compound is capable of increasing UTR-dependent expression of at least one MYC gene.

10. Compound according to claim 1, wherein the tumor is selected among: neuroblastoma, medulloblastoma, retinoblastoma, small cell lung carcinoma, glioma, alveolar rhabdomyosarcoma, primitive neuroectodermal tumor, breast cancer esophageal cancer, cervical cancer, ovarian cancer, head and neck cancer.

11. Compound according to claim 1, wherein the at least one MYC gene is selected among: MYC, MYCN and MYCL genes.
